(19) United States
(12) Patent Application Publication
(10) Pub. No.: US 2007/0082015 A1
(43) Pub. Date: Apr. 12, 2007

(54) Cloning and expression of outer membrane protein C of Salmonella typhi Ty2 and conjugation of the purified insoluble protein to VI-polysaccharide for use as a vaccine for typhoid fever

(75) Inventors: B. L. Jailkhani, New Delhi (IN); M. K. Bhan, New Delhi (IN); R. Kumar, New Delhi (IN); S. Sengupta, New Delhi (IN); Shabirul Haque, New Delhi (IN)

Correspondence Address: THE WEBB LAW FIRM, P.C., 700 KOPPERS BUILDING, 436 SEVENTH AVENUE, PITTSBURGH, PA, 15219, US

(73) Assignees: ALL INDIA INSTITUTE OF MEDICAL SCIENCES, New Delhi (IN); DEPARTMENT OF BIO TECHNOLOGY, New Delhi (IN)

(21) Appl. No.: 11/246,569

(22) Filed: Oct. 7, 2005

(51) Int. Cl. A61K 39/112; C07K 14/255

(52) U.S. Cl. 424/258.1; 530/350

(57) ABSTRACT

The invention relates to a novel carrier protein for use as a vaccine comprising the outer membrane protein C (OmpC) of Salmonella typhi Ty2 for conjugation with VI polysaccharide.

### FIELD OF INENTION

This invention relates to a novel carrier protein and a method of corrugation of the said protein to polysaccharide for use as a vaccine for typhoid.

### BACKGROUND OF THE INVENTION

The surface polysaccharides of several bacterial pathogens are both virulence factors and protective antigens. These are T independent antigens, do not induce an immunological memory or yield a booster response with repeated immunization and are poorly immunogenic in children less than 2 yrs of age (Mond et al, 1995) Conjugation of capsular polysaccharides to a carrier protein renders them immunogenic in infants and capable of eliciting memory, booster responses and isotype switching of antiPS antibodies to igG. There are three vaccines for the prevention of typhoid fever. All these vaccines are moderately effective in 5-18 yrs age group. In view of the fact that the typhoid vaccine has to be administered in children about 1 yr of age, it is necessary to conjugate the Vi polysaccharide with a protein to get the desired results. The technology of conjugation of bacterial polysaccharide to proteins is already available (Kossaczka et al. 1999). The HIB conjugate vaccine is widely used in children loss than one year of age in most developing countries. One such vaccine is already available for typhoid (Lin et al, .2001).

The effective chemotherapy of typhoid fever became possible with the introduction of chloramphenicol (Woodward et al, 1948). When given in proper doses and early in disease, chloramphenicol resulted in rapid clinical cure. Emergence of resistance to chloramphenicol and other anti microbial agents has been a major set back (Mirza et al, 1996). This dimension of the disease Introduces a sense of urgency for focusing on preventive strategies.

Robbins and his colleagues have provided compelling evidence to suggest that surface polysaccharides of several bacterial pathogens are both virulence factors and protective antigens (Schneerson et al, 1992 and 1984; Szu et al, 1983). The examples include capsular polysaccharides of both gram-positive bacteria (Pneumococcus, Streptococcus) and gram negative bacteria (Shigellae and Non-typhoidal Salmonellae) (Pozsgay et al, 1999; Konadu et at. 1996; Robbins et al, 1992). Further, a critical lover of igG antibodies against these polysaccharides may be sufficient to confer immunity. The mechanism of protection relates to igG activated complement mediated bacteriolysis of gram-negative bacteria or bactericidal activity following IgG assisted phagocytosis in gram positives. These bacteria associated with polysaccharide related virulence cause highest Incidence, morbidity and mortality in children, mostly younger than 5 years of age. Infants and young children are also the ones who do not respond immunologically to carbohydrate antigens. Even in adults, being T-independent, carbohydrate antigens induce essentially an IgM response, only a low level of IgG antibodies and no booster response.

The carbohydrates are type 2T independent antigens, which stimulate mature B-cells directly without the intervention of T cell (Mond et al, 1995). In young children with relative immaturity of B-lymphocytes, the carbohydrate antigens are only poorly immunogenic (Kovarik et al, 1998). Further, even in adults in the absence of T-helper activity, the class switching from μ does not take place. Therefore, the antibodies produced are essentially of IgM type with short half-life and inadequate memory (Mond et al, 1995; Kovarik et al, 1998). In order to Increase their immunogenicity and provide T-help, protein/peptide conjugates of carbohydrate antigens have been tried with success. In general, the proteins used for conjugation include cholera toxin, diphtheria toxin, tetanus toxin, meningococcus outer membrane complex and a few others (Szu et al, 1994; Lieberman et al, 1996; Granoff et al, 1993 and Mulholand et at, 1996).

The introduction of technology to conjugate carbohydrate antigens to proteins led to the development of vaccines against Haemophilus infuenzae. Such vaccines have been introduced in infancy in the United States of America and other developed countries. Within a few years, these vaccines have led to almost disappearance of the Hib related diseases in these populations (Rosenstein and Perkins, 2000; Levine et al, 1998 and Santosham, 1993).

Based upon this logic, John Robbins and his colleagues have prepared a conjugate consisting of recombinant exoprotein A of Pseudomonas aefogenosa and Vi capsular polysaccharides. In field testing this vaccine was found to be 91% effective in the prevention of typhoid fever in Vietnam over 27 month follow up (Lin et al. 2001).

The outer membrane protein of Salmonellae has been shown to be protective by various groups in experimental and clinical typhoid fever. The outer membrane proteins (OMPs) in gram-negative bacteria are generally known to be highly immunogenic molecules. Studies in mice have shown that immunization with OMPs form Salmonella typhi, Nesseria gonorrhea, Niesseria meningitides. Haemophilus influenzae and many other pathogens resulted in protection against infections caused by these bacteria (Peters et al. 1999; Armando et al, 1988; Nandakumar, 1994; Muthukkaruppan el al 1992; Singh et al, 1999). Recently, in a human study, it was reported that Hib polysaccharide conjugated with outer membrane protein complex (PRP-Omp) of Niesseria meningitides induced long lasting protective antcapsular antibodies; one dose of PRP-Omp polysaccharide complex was able to induce immunologic memory in infants (Bulkow et al, 1993 and Kurikka et al 1995).

In Salmonella, a major class of outer membrane proteins coded for by the Omp-F and Omp-C genes is known as porins because, they produce relatively nonspecific pores or channels that allow the passage of small hydrophilic molecules. The abundance of these two Omps is a function of the growth medium. Omp-F predominates in low osmolarity whereas Omp-C predominated when the osmolarity is increased. In addition, low temperature, which is typical of the free-living environment, also triggers a high Omp-F (Puente et al, 1991). The osmolarity of intestinal compartment is high as compared with the aqueous environment that these organisms encounter in the free-living state. The choice of Omp-C of Salmonella typhi as a carrier molecule for Vi antigen will therefore be most appropriate.

### OBJECTS OF THE INVENTION

An object of this invention is to prepare a novel carrier protein for conjugation with Vi, PCR amplification, expression and purification of the OmpC gene from Selmonella typhi Ty2.

Another object of this invention is to prepare a method for conjugation of the carrier protein with Vi capsular polysaccharide.

Further object of this invention is to prepare a vaccine for Typhoid in children below 2 yrs of age.

### BRIEF DESCRIPTION OF THE INVENTION

According to this invention a novel carrier protein for use as a vaccine comprising the outer membrane protein C, (OmpC) of Saklmonellae typhi Ty2 for conjugation with VI polysaccharide.

According to this invention there is also provided a method for conjugation of carrier protein with polysaccharide comprising, purifying said carrier protein by solubilization from inclusion bodies, subjecting the soluble protein to the step of activation; subjecting the said protein to the step of conjugation with VI polysaccharide in presence of Urea; dialysing the conjugate in against buffer; separating the unconjugated protein by centrifugation.

### DETAILED DESCRIPTION OF THE INVENTION

The novelty of the present invention preparation lies in the fact that;
1) Cloning and expressing the Outer Membrane Protein C. (OmpC) and using it as a carrier for conjugation with Vi polysaccharide also from Salmonella typhi Ty2 for prevention of typhoid fever.
2) OmpC is a fairly conserved protein in Enterobacteriaceae more so within Salmonellae and protective independent of VI antigen. The use of this protein as a carrier is an added advantage in the indian subcontinent as it may take care of the 25% cases of enteric fever which are caused by Salmonella paratyphi A in this region.
3) A method has been developed for conjugation to suit use of insoluble proteins as carriers by modifying the method described by Kossaczka et al, 1999. OmpC used as a carrier protein is purified by solubilization from inclusion bodies. The pure protein precipitates on storage at 4° C. and is made soluble in 8M urea. Activation with adipic acid dihydrazide and subsequent conjugation with VI are carried out in presence of urea. The conjugate is soluble in aqueous buffer and the unconjugated protein is recovered by a simple contrifugation step. This unconjugated protein can be reused for conjugation thus preventing loss and therefore reducing the cost of preparation of the carrier protein.

In order to clone and expess a novel carrier protein for conjugation with VI, PCR amplification of the OmpC gene was done and the amplicon was cloned in the expression vector pPROEX HT (invitrogen). The protein was expressed in E. coli DH5 a cell line and purified from the inclusion bodies through a NI-NTA column under denaturing conditions using standard protocols. Conjugation of the denatured OmpC with Vi capsular polysaccharide was carried out in the following two steps,

STEP I: Activation of the OmpC Protein with Adipic Acid Dihydrazide:

1) The protein was insoluble in water and hence was suspended in PBS (pH 6.8) with 0.25 mM sodium phosphate buffer containing 8M urea.
2) MES buffer 0.5M pH 5.6 was added to the protein suspension with constant stirring. The pH of the resultant solution was 5.7.
3) Five times excess of adipic acid dihydrazide and EDC (10 mM) was added to this solution which was constantly mixed on a roller mixer for 1 hr. The final pH of the solution was 5.0.
4) AH-protein was extensively dialyzed overnight at 4° C. against PBS pH 6.8 with 0.25 mM sodium phosphate buffer. On removal of urea the protein precipitates out. Protein was therefore solubilized by gradually increasing the amount of urea to a final concentration of 8M. Alternatively, for smaller volumes, the protein can be dialyzed against PBS pH 6.8 with 0.25 mM sodium phosphate buffer containing 8M urea thus bypassing the resuspension step. Estimation of protein was done by method of Lowry et al, 1951 and the Vi polysaccharide was indirectly estimated by determination of the O-acetyl content of the corrugate (Hestrin, 1949).
   STEP II: Conjugation of VI Polysaccharide to OmpC-AH:
1. The VI polysaccharide in PBS was mixed with 0.5M MES pH 5.6 at room temperature.
2. While the mixture was being stirred, EDC was added followed by dropwise addition of OmpC-AH. The volumes were so adjusted that the concentration of OmpC-AH and Vi polysaccharide was equal and the conc. of EDC was 10 mM.
3. The reaction was allowed to continue for 3 hrs at room temperature with constant mixing after which the pH was raised to 7 with 1.0M sodium phosphate buffer pH 7.2 and the mixture was stored overnight at 4° C.
4. The mixture was extensively dialyzed against 0.15M saline containing 0.25 mM sodium phosphate buffer pH 7.0. The Vi-OmpC conjugate was soluble while the unconjugated protein precipitated out.
5. The unconjugated protein was removed by centrifugation at 10,000 g and can be reused for conjugation.

6. O-acetyl content of the conjugate was measured by the Hestrin's (1949) method and the Vi content was calculated from it.
7. The urea content in the supernatant which was used for immunizing mice was 0.08 mg %.
8. The ratio of polysaccharide to protein in the final preparation of conjugate was 1:1.42.

### EXAMPLE

In order to test the immunogenicity of this conjugate, 30 mice each were Immunized with VI-OmpC protein alone. Ten mice were injected with saline only. All mice were given subcutaneous injections of antigen in 0.1 ml saline divided at 4 sites. The primary immunization was followed by 2 booster injections on day 14 and day 21.

Each mice in the group that was injected with the conjugate received 5 ug and 7.1 ug of VI and OmpC respectively (In each injection).

The result of antibody estimation by ELISA are as follows;

Group-I Saline Control.

| | Anti-whole | | Anti-IgG | |
|---|---|---|---|---|
| | Mean ± S.D | Responders | Mean ± S.D | Responders |
| 1^{st} Injection | 0.2 ± 0.00 | 0/10 | 0.16 ± 0.00 | 0/10 |

Group II (Conjugate)

| | Anti-whole | | Anti-IgG | |
|---|---|---|---|---|
| | | Respond- | | Respond- |
| | Mean ± S.D | ers | Mean ± S.D | ers |
| 1^{st} Injection | 5.48 ± 8.87 | 8/9 | 15.32 ± 32.31 | 8/9 |
| 2^{nd} Injection | 10.90 ± 19.04 | 9/10 | 18.51 ± 23.37 | 9/10 |
| 3^{rd} Injection | 14.94 ± 11.79 | 10/10 | 35.75 ± 23.24 | 10/10 |

Group III (Vi—alone)

| | Anti-whole | | Anti-IgG | |
|---|---|---|---|---|
| | Mean ± S.D | Responders | Mean ± S.D | Responders |
| 1^{st} Injection | 0.223 ± 0.056 | 3/10 | 0.16 ± 0.00 | 0/10 |
| 2^{nd} Injection | 0.265 ± 0.103 | 5/10 | 0.16 ± 0.00 | 1/10 |
| 3^{rd} Injection | 0.61 ± 0.65 | 7/9 | 1.65 ± 3.46 | 2/9 |

Group—IV (OmpC)

| | Anti-whole | | Anti IgG | |
|---|---|---|---|---|
| | Mean ± S.D | Responders | Mean ± S.D | Responders |
| 1^{st} Injection | 0.2 ± 0.00 | 0/10 | 0.16 ± 0.00 | 0/10 |
| 2^{nd} Injection | 0.2 ± 0.00 | 0/10 | 0.16 ± 0.00 | 0/10 |
| 3^{rd} Injection | 0.2 ± 0.00 | 0/10 | 0.16 ± 0.00 | 0/10 |

Vi antibody titres were expressed as geometric mean (GM) with respect to a reference mouse serum assigned an arbitrary value of 100 for total antibodies and 66 for anti igG antibodies at 1:100 dilution of sera.

We claim:

1. A novel carrier protein for use as a vaccine comprising the outer membrane protein C (OmpC) of Salmonella typhi Ty2 for conjugation with VI polysaccharide.

2. The carrier protein as claimed in claim 1, wherein OmpC is a fairly conserved protein in Enterobacteriaceae within Salmonellae independent of VI polysaccharide antigen.

3. A method for conjugation of carrier protein with polysaccharide comprising;
purifying said carrier protein by solubilization from inclusion bodies,
subjecting the soluble protein to the step of activation;
subjecting the said protein to the stop of conjugation with Vi polysaccharide in presence of Urea;
dialysing the conjugate in against buffer;
separating the unconjugated protein by centrifugation.

4. The method as claimed in claim 3, wherein said purified protein does not stay in solution for long and precipitates on storage overnight at 4° C. and requires 8M urea for solubilization.

5. The method as claimed in claim 3, wherein the protein in activated with adipic acid dihydrazide

6. The method as claimed in claim 3, wherein the buffer used is sodium phosphate pH 7.0.

7. The method as claimed in claim 3 where in the conjugate is purified from the unconjugated protein by a one step process.

8. The method as claimed in claim 3, wherein the ratio of polysaccharide to protein in the conjugate is 1:1.42.

9. The method as claimed in claim 3, wherein the said unconjugated protein is roused for conjugation to prevent loss.
